(19) United States
(12) Patent Application Publication
(10) Pub. No.: US 2024/0366282 A1
(43) Pub. Date: Nov. 7, 2024

(54) PLASMA INSTRUMENT WITH LATERAL WINDOWS

(71) Applicant: Erbe Elektromedizin GmbH, Tuebingen (DE)

(72) Inventors: Martin WALZ, Rottenburg (DE); Sandra MOSER, Albstadt-Laufen (DE); Johannes HEYM, Tuebingen (DE); Fabian BEUTLER, Neckartenzlingen (DE)

(21) Appl. No.: 18/642,171

(22) Filed: Apr. 22, 2024

(30) Foreign Application Priority Data
May 4, 2023 (EP) 23171492.4

(51) Int. Cl. A61B 18/04; A61B 18/00

(52) U.S. Cl. CPC A61B 18/042; A61B 2018/00077; A61B 2018/00101

(57) ABSTRACT

An instrument for plasma treatment of biological tissue comprises plasma exit windows in the form of openings having an edge extending obliquely relative to the longitudinal direction (L), which can be formed by flank of a web. The webs taper in radial direction (+R) and are preferably obliquely inclined relative to the longitudinal direction (L). The webs support a proximal section of the instrument head and connect the latter seamlessly monolithically with a proximal part of the head. Due to the outward tapering of the webs, their shading effect is minimized or eliminated.

This application claims priority to European Patent Application No. 23171492.4, filed May 4, 2023, the entirety of which is incorporated herein by reference.

The invention refers to a plasma instrument for influencing biological tissue, particularly for surgical treatment of human or animal tissue. The plasma instrument is configured as laterally or radially operative instrument.

Plasma instruments for influencing biological tissue are known from the prior art. For example, DE 198 20 240 A1 as well as EP 1 297 082 A1 respectively disclose instruments having a hose-like base body through which a lumen extends from its proximal end to its distal end on which a head with one or multiple lateral windows is arranged. An electrical conductor extends through the lumen, which is connected to a central electrode at its distal end. In operation an electrode connected to an HF-voltage source ionizes the flowing gas, which then exits laterally as plasma stream. In an embodiment two slit-like plasma exit windows are provided that are axially arranged at different locations and slightly overlap with one another.

EP 3 831 291 A1 discloses an instrument for production of a plasma for influencing biological tissue. Again, the instrument comprises a hose-like body, which, at least in one embodiment, supports a head having a lateral opening on its distal end through which a plasma stream may exit.

Instrument for creation of a radially operative plasma stream are also known from EP 1682 023 A1 as well as U.S. Pat. No. 9,510,889 B2. These instruments comprise an axial opening at their distal end out of which an electrode projects. The electrode supports an insulating body at its distal end, which limits a ring-shaped radially opening slit with the remaining instrument. The plasma stream has unblocked radial exit around 360° at each circumferential location, wherein however the insulating body has to be supported by the electrode and has to be maintained in a stable manner.

Additional prior art can be taken from US 2021/0259756 A1, EP 3 422 981 A2, EP 3 372 183 A1, JP 2002-301088 A and GB 2 573 128 A.

It is the object of the invention to provide a robust plasma instrument with lateral exiting plasma stream.

This object is solved by a plasma instrument according to claim 1:

The plasma instrument according to the invention comprises a hose- or tube-like body at the distal end of which a head is provided having at least two openings, which are connected with a lumen extending through the hose-like body. The openings are separated by a web having a cross-section tapering outwardly in a radial direction. Preferably in the head in the proximity of the opening an electrode is arranged, which is configured to emit an electrical current suitable for ionization of a gas stream. The electrode thereby preferably comprises a diameter that is larger than a width of the web to be measured in a circumferential direction at its side facing the electrode. Due to this measure and due to the outward tapering of the web, the ability of the web is minimized to shade or impede a plasma stream. The plasma stream can thereby flow through both openings adjoining the web, can switch from one opening to the other in an unimpeded manner and, if it flows through both openings, join behind the web. The treatment of biological tissue is thereby simplified. A surgeon can guide the plasma instrument according to the invention and the plasma stream exiting therefrom as simple as a plasma instrument having a ring-shaped uninterrupted radial plasma outlet.

The outwardly tapering web can be configured triangularly, trapezoid shaped, with straight or rounded edges as well as with pointed or rounded corners in cross-section. Thereby, preferably, the width of the web measured in circumferential direction is larger than the width of the adjoining opening also measured in circumferential direction. In addition, the web is (the webs are) preferably slim, i.e. their length measured from their proximal to their distal end is longer than their half circumference. It is, however, also possible to provide one or more webs on the head, which do not taper outwardly. This can apply to one web, some of the webs or all of the webs.

In a preferred instrument the openings form a line extending around the entire head, wherein the openings are separated by webs from one another. The webs support the distal end of the head, which is preferably configured seamlessly monolithically from a temperature-resistant material, such as ceramic.

The webs separating the openings from one another are preferably orientated obliquely to the longitudinal direction of the plasma instrument, i.e. arranged inclined in circumferential direction. In doing so, the openings are, for example, trapezoid-shaped or also diamond-shaped with straight or rounded edges as well as with straight or rounded corners. Preferably the webs are inclined in circumferential direction with alternating sense of direction, so that the openings form a line of triangular-shaped windows, the tips or narrow ends of which are alternatingly facing in distal direction (positive longitudinal direction) or proximal direction (negative longitudinal direction).

Due to this measure, the openings can overlap one another in circumferential direction as well as in longitudinal direction. Thereby and due to the radially outwardly tapering cross-section of the webs, the continuity of the plasma stream can be achieved, if it changes from one opening to the adjacent opening when flowing to the next tissue section.

Additional advantageous details are subject matter of the drawing, the associated description or of claims. The drawing shows:

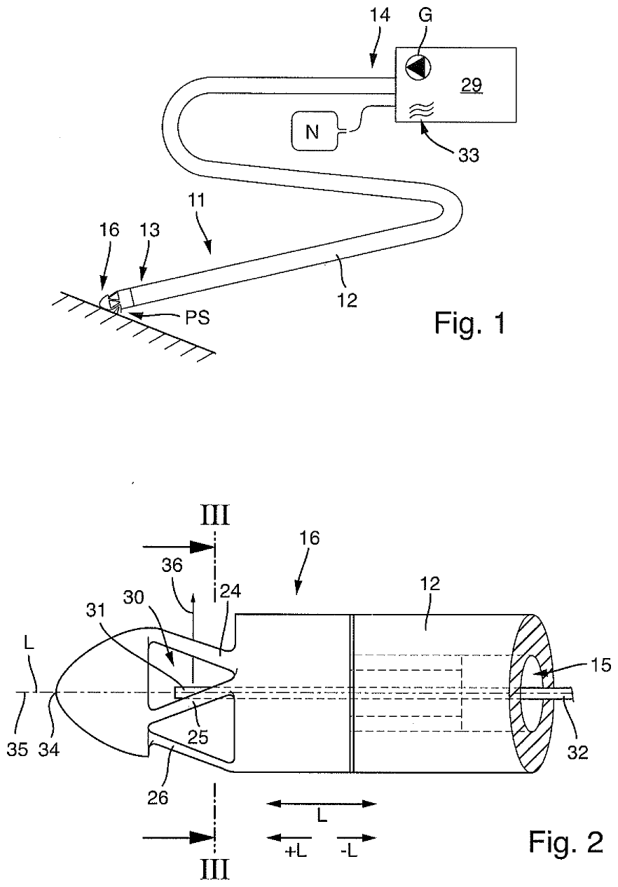

FIG. 1 illustrates a plasma instrument according to the invention connected to a supplying apparatus in a perspective illustration,

FIG. 2 illustrates a distal end of the plasma instrument according to FIG. 1 in enlarged lateral view, however, not drawn to scale,

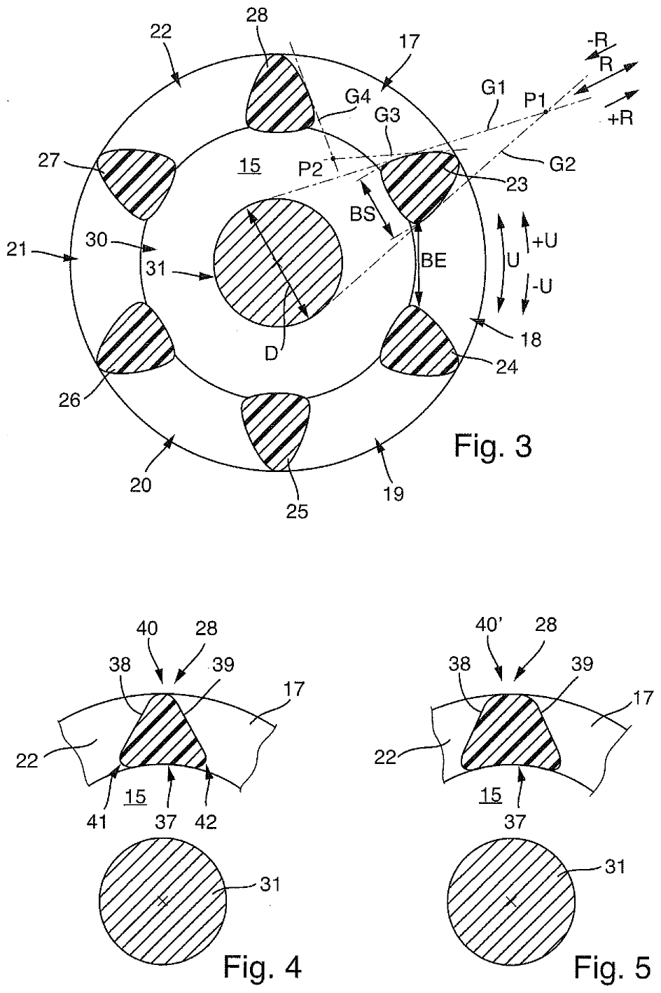

FIG. 3 illustrates the plasma instrument according to FIGS. 1 and 2 cut along the cutting line III-III in FIG. 2,

FIG. 4 illustrates a modified embodiment of the plasma instrument according to FIGS. 1 and 2 showing a portion of the device cut along the cutting line III-III in FIG. 2, highly enlarged,

FIG. 5 illustrates a modified embodiment of the plasma instrument according to FIGS. 1 and 2 showing a portion of the device cut along the cutting line III-III in FIG. 2, highly enlarged,

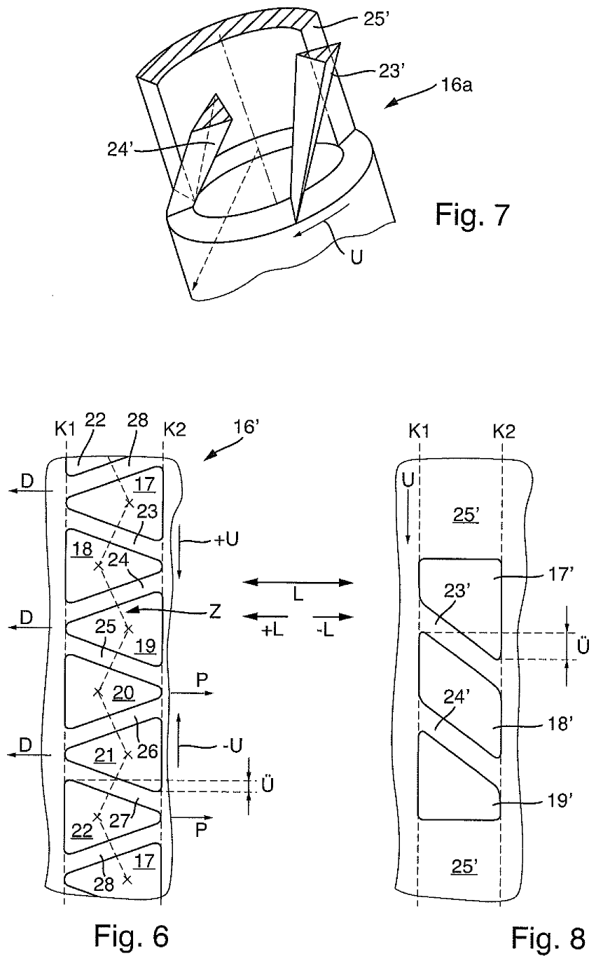

FIG. 6 illustrates an unwrapped depiction of the head from FIG. 2 for illustration of the geometric relations,

FIG. 7 illustrates a modified embodiment of a plasma instrument according to the invention in a cut perspective illustration in part,

FIG. 8 illustrates an unwrapped depiction of the head according to FIG. 7 for illustration of the geometric relations,

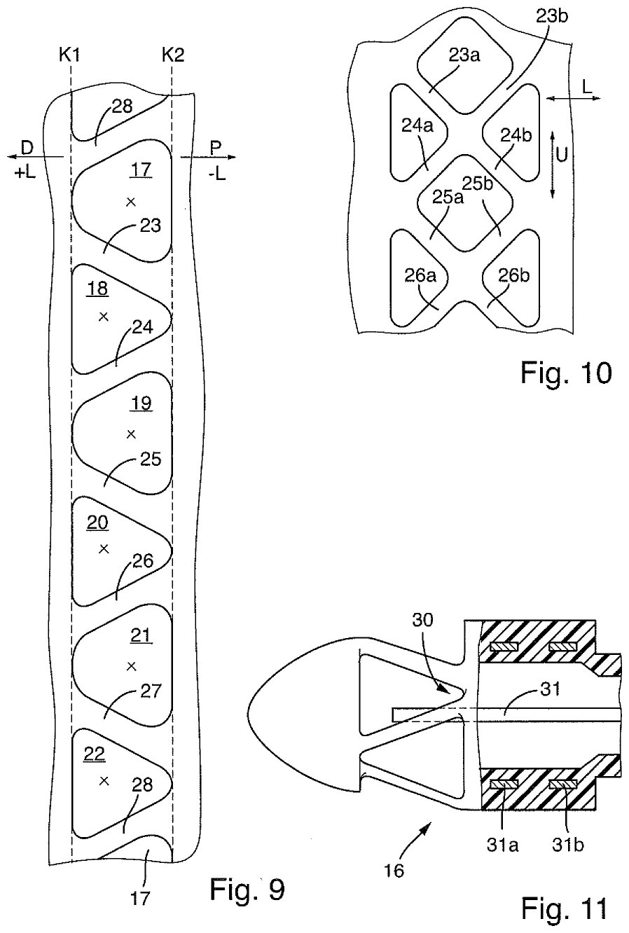

FIGS. 9 and 10 illustrate unwrapped depictions of additional alternative embodiments of probes according to the invention and

FIG. 11 illustrates a plasma probe having at least two electrodes in an illustration of its head in part and partly cut.

FIG. 1 illustrates a plasma instrument 11 configured as flexible probe having a flexible hose as body 12. The body 12 comprises a distal end 13 and a proximal end 14 as well as a lumen 15 extending from the proximal end 14 to the distal end 11. The body 12 can comprise one single lumen 15, as illustrated in FIG. 2, or also multiple lumen. The body 12 is configured in a bendable or flexible manner, for example in order to be moved as probe through the working channel of an endoscope to the operation site of a patient. If the endoscope comprises a controllable end, the flexibility of the instrument allows to carry out a respective bending movement.

The instrument 11 can not only be provided as endoscopic usable probe, but also in another form, for example as laparoscopic instrument, whereby the body 12 is a rigid tube. Also, the instrument 11 can be configured for the open surgical use having a short body 12 and a hand piece provided at the proximal end.

On the distal end 13 of body 12 a head 16 is arranged, which comprises at least two, preferably however multiple openings, for example six openings 17 to 22, as apparent from FIG. 3. Alternatively, also three, four, five, seven, eight or more openings can be provided. The openings 17 to 22 are separated by webs 22 to 28 from each other, which consist of heat resistant material, for example ceramic, such as preferably the entire head 16. The webs 23 to 28 are slim, i.e. their length measured in longitudinal direction L is longer, preferably multiple times longer, than their width measured in circumferential direction U.

At the proximal end 14 lumen 15 is connected to an apparatus 29, which serves for supply of instrument 11. For example, the apparatus 29 can be configured as gas source G or can comprise such a gas source. For this purpose, it can be connected to a gas storage, for example a gas bottle or the like, and can control, particularly for releasing and blocking, the gas flow, with which the lumen 15 is supplied. The gas storage can contain particularly argon or also another inert gas, particularly an inert gas suitable for plasma creation. As necessary, apparatus 29 can also be configured for supply of active gases, i.e. reactive gases, aerosols, vapors or the like.

The lumen 15 is connected with the openings 17 to 22, so that gas flowing through the lumen to the head 16 can exit equally from all openings 17 to 22. This also applies if multiple lumen are provided in the body 12 parallel to one another.

The instrument 11 comprises in addition at least one plasma creation device 30, for example in the form of an electrode 31, the distal end of which is positioned in the region of the openings 17 to 22. The electrode can be formed by a substantially cylindrical body of metal, the distal end of which is arranged in the region of the openings 17 to 22, preferably substantially in the center thereof. As illustrated, electrode 31 can have a circular cross-section or also a polygonal cross-section.

An electrical conductor 32 can extend from the electrode 31 up to the proximal end 14 of instrument 11 and can be connected to the apparatus 29 there. The apparatus 29 can comprise an electrical source, for example in form of a high voltage high frequency generator 33, via which the electrode 31 can be supplied with an electrical voltage. The high frequency voltage has such an amount that it is sufficient for ionization of the gas stream at the electrode 31 supplied via lumen 15 in order to form a plasma. The voltage usually has an amount of multiple 100 Volts at a frequency above 100 kHz, preferably multiple 100 kHz, further preferably however below 5 MHZ.

The special feature of the invention is the configuration of head 16, which has at least preferably a cross-section tapering toward the distal tip 34. The openings 17 to 22 and thus also the webs 23 to 22 can be entirely or partly arranged in the tapering section of head 16, whereby the tapering section starts in FIG. 2 approximately at cutting line III-III. In the tapering section the diameter of head 16 decreases continuously up to the distal tip 34 where the head 16 ends in a curvature, preferably bluntly.

The individual openings 17 to 22 are preferably separated by webs 23 to 28, which are arranged inclined relative to a longitudinal direction L at least in circumferential direction U. The longitudinal direction L extends longitudinally through lumen 15 and is indicated in FIG. 2 by a longitudinal center axis 35, which forms a symmetry axis for the rotational symmetry of the head 16. The rotational symmetry is three-fold in the embodiment according to FIGS. 2 and 3, i.e. in case of a rotation of the head about 120° around the longitudinal center axis 35, the rotated head 16 is congruent with the non-rotated head 16.

The openings 17 to 22 limited between the webs 23 to 28 are orientated radially relative to the longitudinal center axis 35. In FIG. 2 the opening direction of the window 17 is marked by means of a dashed arrow 36. The openings 17 to 22 thus open transverse to the longitudinal center axis 35 and thus also transverse to the longitudinal direction L in radial direction R or, as indicated by arrow 36, also slightly inclined relative to the radial direction R. The radial direction R is orientated orthogonal to the longitudinal direction L. For determination of the opening direction, head 16 can be first considered without window as rotational body tapering toward the distal tip 34. As illustrated by FIG. 2, the tapering can increase toward the distal tip 34, so that the longitudinal section through the head 16 approximates a parabolic shape or a half ellipse. If on such a virtual head 16 without opening, an opening is drawn at the position provided for the respective opening 17 to 22 and if in the center of the area thereof the normal vector is indicated, the latter characterizes the direction of arrow 36.

The webs 23 to 28 have cross-section tapering in positive radial direction +R (radially outwardly). This cross-section is illustrated in FIG. 2 in a thin-thick-cross-hatched manner. Preferably, the cross-section is triangular, as apparent from FIG. 3 and illustrated by way of example of web 28 in FIG. 4. The web 28 is cut in the same cutting plane III-III, as indicated in FIG. 2 and illustrated in FIG. 3. Due to the thickness reduction of webs 23 to 28 radially outwardly, the window cross-section of the openings 17 to 22 increases radially outwardly.

The cross-section of web 28 illustrated in FIG. 4 representatively for all webs 23 to 28 comprises a lateral surface 37 facing the longitudinal center axis 35 as well as two additional lateral surfaces 38, 39, laterally limiting the openings 22 and 17. The cross-section of the webs 23 to 28 are preferably substantially constant along each web 23 to 28. The lateral surfaces 38, 39 (FIG. 3) of web 23 configured in a planar or also convexly rounded manner limit an acute angle with one another, which opens toward the longitudinal center axis. Thereby web 28 faces electrode 31 with its interior lateral surface 37. With its preferably rounded corner 40, web 28 points radially outwardly with view from the longitudinal center axis 35, i.e. away from the longitudinal center axis 35. The remaining corners 41, 42 are preferably rounded as well. The lateral surfaces 38, 39 can be planar or preferably slightly convexly rounded. The lateral surface 37 can be planar or as illustrated in FIG. 4 concave, as necessary however also convexly rounded.

FIG. 5 illustrates a modified embodiment of the plasma probe according to the invention in which the web cross-section is not triangular, different to FIG. 4, but configured in a trapezoid-shaped manner. Instead of the rounded corner 40, now a straight or rounded edge 40′ is provided. Apart therefrom, the above explanations with regard to the lateral surfaces 38, 39 as well as the interior surface 37 apply accordingly.

It is common to all embodiments provided so far that the electrode 31 has a diameter D, see FIG. 3, which is preferably larger than the web width BS, which is to be measured at the widest position of each web 23 to 28 in circumferential direction U respectively. In doing so, a minimization of the shading of electrode 31 to the outward is achieved. If the straight lines G1, G2 illustrated in FIG. 3 are drawn so that they touch electrode 31 at opposite sides on one hand and the web 23 at its lateral surfaces 38, 39 on the other hand, these straight lines G1, G2 intersect at a point P1 outside head 16, wherein the respective web 23 is positioned between the point P1 and the electrode 31. If the straight lines G3 and G4 are arranged in a manner touching the lateral surfaces of webs 23, 28 limiting window 17, these straight lines G3, G4 intersect inside head 16, preferably in an interstice between the electrode 31 and the webs 23, 28. If the electrode 31 comprises a polygonal cross-section instead of a circular cross-section, the diameter D is replaced by the diagonal of the polygonal cross-section. The electrode 31 can be configured as individual electrode with round or polygonal cross-section. The electrode 31 can also be formed by a bundle of individual electrical conductors, which are arranged with distance to one another or in contact to one another. Then the diameter D characterizes the outer diameter of the cross-section of the bundle of conductors.

The width BS of web 23 is preferably smaller than the width BE of at least one adjacent opening, in FIG. 3 the opening 18, also measured in circumferential direction U. This applies at least at one axial position, whereby the width BS and the width BE are measured at the same axial position. This also guarantees the continuity of the plasma stream, which is therefore neither interrupted by web 23 nor by the other webs 24 to 28. The indicated condition for the widths BS and BE (BE>BS) respectively applies at least where the openings have their largest extension in circumferential direction U.

FIG. 6 illustrates an unwrapped depiction 16′ of head 16, whereby the ends of the unwrapped depiction overlap. For example, the six windows 17 to 22 are triangular. Adjacent webs 23 to 28 are inclined in positive circumferential direction +U and negative circumferential direction-U oppositely in pairs respectively. Thereby the absolute values of the inclination angles, which the individual webs 23 to 28 limit with the circumferential direction U are equal. In other words, the inclination angle of each web 23 to 28 changes its sign along the circumferential direction from web to web. In doing so, the openings 17 to 22 obtain a triangular basic shape with more or less strongly rounded corners, wherein the bases of the triangles marked by openings 17 to 22 are positioned on circles K1, K2 that are parallel to one another having equal or slightly different diameters. The center points of the circles K1 and K2 are positioned on the longitudinal center axis 35. The tips of the triangles marked by openings 17 to 22 alternatingly point in distal direction D (i.e. positive longitudinal direction +L) or proximal direction P (i.e. negative longitudinal direction-L). The substantially triangular openings 17 to 22 have centers of area, which can be positioned on a zigzag line Z, as indicated in FIG. 5.

The inclinations or inclination angles of webs 23 to 28 are dimensioned so that the openings 17 to 22 overlap in circumferential direction U. The circumferential direction is characterized by the progress of the circles K1, K2 illustrated here as straight lines, due to the unwrapped depiction. In FIG. 5 the overlap is illustrated by way of example of web 27 and the openings 21, 22. The corner of opening 21 positioned in front in positive circumferential direction +U and the opening of corner 22 located at the rear in positive circumferential direction +U overlap one another about the overlap absolute value U. The latter is at least zero and preferably larger than zero. In other words, if opening 21 is virtually moved in distal direction D and/or opening 22 is virtually moved in proximal direction P, the openings 21, 22 touch or overlap one another.

In the embodiment according to FIGS. 1 to 6, webs 23 to 28 are in pairs inclined in and against the circumferential direction (i.e. in +U and −U). It is however also possible to incline webs 23′, 24′ in the same orientation as illustrated based on an embodiment according to FIGS. 7 and 8. While instrument 11 described above is an instrument with 360°, all-round effectiveness instrument 11′ according to FIG. 7 is provided for tissue treatment only in a limited circumferential area. The head 16a comprises a wider section 25′, which is configured with or without inclination in circumferential direction and can be considered as curved wall. Three openings 17′, 18′, 19′ are obtained, which can be configured trapezoid-shaped or diamond-shaped. Trapezoid-shaped opening 17′ and diamond-shaped opening 18′ overlap in circumferential direction U again. The same applies for the opening 18′ and the trapezoid-shaped opening 19′. Apart therefrom, the above description applies on the basis of the already introduced reference signs.

Further modifications are possible. FIG. 9 therefore shows an unwrapped depiction of a modified head 16 for which the description of the embodiment according to FIGS. 1 to 6 applies fully accordingly apart from the special features indicated in the following:

At least some of the openings 17 to 22 or all of the openings 17 to 22 are not configured in triangular, but trapezoid shaped manner. For this purpose, instead of the respective tip of each surface 17 to 22 pointing in distal direction D or proximal direction P, a strong rounding or also a short edge is provided, which transitions into the respective web 23 to 28 by means of two curvatures.

The embodiments described so far use openings 17 to 22 arranged between the two virtual circles K1, K2 in one single line. However, also arrangements in multiple lines are possible, as illustrated in FIG. 10. The special feature of this arrangement consisting in total of three lines of rounded triangular or quadrangular openings are again inclined webs 23a, 23b, 24a, 24b, 25a, 25b etc., which are inclined in or against circumferential direction U and are thus arranged non-parallel to the longitudinal direction L. Again, the individual openings overlap in circumferential direction with one another.

Another modification is possible with regard to the configuration of the plasma creation device 30. In the embodiments described so far only one blank electrode 31 is provided as plasma creation device 30 in order to create a largely thermal plasma. These are monopolar instruments in which an electrical current flows from the electrode 31 through the plasma to the tissue and therefrom back to the generator 33 via a neutral electrode N. In all of the above-described embodiments alternatively also one or multiple electrodes 31a, 31b arranged in electrically insulated manner can be provided for forming a barrier discharge. For example, the electrode 31a and/or 31b can be embedded as bowl- or ring-shaped electrode in the material of head 16 or can be arranged in another insulated manner therein. The two poles of the generator can be connected with the insulated electrodes 31a, 31b in order to create a barrier discharge. In this case the electrode 31 can be omitted. Alternatively, one or both electrodes 31a, 31b can be connected to one pole of the generator and the electrode 31 that is uncovered or provided with an insulating layer can be connected with the other pole of the generator in order to create a barrier discharge. Such arrangements particularly serve for creation of non-thermal warm or cold plasma.

The different plasma creation devices 30 described briefly herein can be combined with any of the above-described heads according to FIGS. 1 to 9.

The dimensional relations described with regard to FIG. 3 according to which the diameter D of electrode 31 is at least as large and preferably larger than the width BS of web 23 (and also additional webs) is also advantageous in probes with lateral windows according to the previous and the following description in which, however, web 23 (and also additional webs) do not taper outwardly. For example, such webs can have a square or rectangular cross-section with straight or rounded edges and pointed or rounded corners. Also, the webs can have a round cross-section.

The instrument 11 described so far operates as follows:

In operation instrument 11 is supplied with gas, for example argon, from the apparatus 29 so that it flows through lumen 15 and out of openings 17 to 22. In addition, the electrical source 33 is activated so that an electrical discharge occurs for ionization of the gas stream and for creation of plasma at the plasma creation device 30. Particularly in the monopolar variant in which one pole of the electrical source 33 is connected with the preferably uncovered electrode 31 and the other pole of the source 33 is connected with the patient, an electrical discharge forms between the electrode 31 and the biological tissue closest to the head 16, so that a plasma stream PS is established through the opening positioned inbetween, for example opening 19. This condition is illustrated in FIG. 1.

If now instrument 11 is moved, it can happen that the discharge, i.e. the plasma stream, finds its way rather through the adjacent window 18 or 20. The transition of the plasma stream from window 19 to window 18 or 20 is thereby continuous and smooth, because the windows overlap in circumferential direction with one another, particularly with viewing direction in longitudinal direction L, as obvious by way of example of FIG. 5. The obliquely orientated and radially outwardly tapering webs 23 to 28 do therefore not block the discharge, which remarkably reduces the volatility of the plasma jet, recognized as spark by the treating person, compared with instruments having webs orientated in longitudinal direction.

If a device with barrier discharge is used as plasma creation device 30 as illustrated according to FIG. 10, it can be achieved that the plasma is created in a ring-shaped area covering all of the openings 17 to 22 without gap, whereby shadings due to webs 23 to 28 are again minimized.

An instrument 11 according to the invention for plasma treatment of biological tissue comprises plasma exit windows in form of openings 17 to 22 having an edge extending obliquely relative to the longitudinal direction L, which can be formed by flank 38, 39 of a web. The webs 23 to 28 taper in radial direction +R and are preferably obliquely inclined relative to the longitudinal direction L. The webs 23 to 28 support a proximal section of the instrument head 16 and connect the latter seamlessly monolithically with a proximal part of the head 16. Due to the outward tapering of the webs, their shading effect is minimized or eliminated.

1. A plasma instrument for influencing biological tissue, comprising:
a longitudinal body configured as a hose or a tube comprising a distal end and a proximal end,
a lumen extending along a longitudinal direction (L) from the distal end to the proximal end, and configured to be connected to a gas source (G),
a head connected to the distal end, and the head comprising two openings connected to the lumen such that the two openings separated from one another by a web, which has a cross-section tapering outwardly in a radial direction (R).

2. The plasma instrument according to claim 1, further comprising an electrode arranged in the longitudinal body or the head, and the electrode is configured to be connected to an electrosurgical generator (EG) by an electrical conductor extending toward the proximal end.

3. The plasma instrument according to claim 2, wherein the electrode has a diameter (D) and the web has a width (BS) at a side facing the electrode, wherein the width (BS) is smaller than the diameter (D) of the electrode.

4. The plasma instrument according to claim 2, wherein the electrode has a circular cross-section.

5. The plasma instrument according to claim 1, wherein the web has a planar, convex, or concave shape at a side facing the electrode.

6. The plasma instrument according to claim 1, wherein the cross-section of the web is trapezoid-shaped or triangular.

7. The plasma instrument according to claim 1, wherein the cross-section of the web comprises rounded corners.

8. The plasma instrument according to claim 1, wherein the web and the openings have a widths (BE, BS),
respectively, in a circumferential direction (U) that is orientated orthogonal to the longitudinal direction (L) as well as to the radial direction (R), wherein the width (BE) is larger than the width (BS) of the web.

9. The plasma instrument according to claim 1, characterized in that the head is seamlessly monolithically formed from a temperature-resistant material.

10. The plasma instrument according to claim 1, wherein the openings formed in the head form a perimeter extending around the entire head.

11. The plasma instrument according to claim 1, wherein the web is orientated obliquely to the longitudinal direction (L).

12. The plasma instrument according to claim 10, further comprising multiple webs arranged between the openings, which are inclined in circumferential direction (U) with alternating sense of direction (+U, −U).

13. The plasma instrument according to claim 10, wherein the openings are configured in a manner tapering in longitudinal direction (L) with alternating sense of direction (+L, −L).

14. The plasma instrument according to claim 10, wherein the openings are configured such that a portion of one opening overlaps with a portion of an adjacent opening in circumferential direction (U) and in longitudinal direction (L).

15. The plasma instrument according to claim 10, characterized in that the openings are trapezoid- or triangular-shaped with rounded corners.
